SEQUENCE LISTING (8 sequences)

SEQ ID NO: 1 - DNA, 3930 bp, Homo sapiens
tgcaatgctagccccttcaagtaggtggccccggagtgcacagctggcctcagcagacaggcggttctctttctctcttttcttcccttctacctttcctttccttcctttccttccttctcctttctctccttctctttatttctcccctttcttccttcctttctccctttattacttttccttccttcttcttccctttcttttttcttttctttctttccttccttctttctctttctcctcctctctgtctttccatttgccttcatccttttgcagcaactaagattcaaacttcataaacttttggccaggtgcagtggcctgtaataacagcactttgagaggcaaagacagaaggatcccttgaggccaggagttggagaacagcctgggcaacatagcaagatcccacttctaaaaaaagtaaataattttttttaattagccagacgtggtggcacatgtctgtggtctgagctgcataggaagcagaggtgggagaactgcttgagcccaggagctcgaggctgcagtgggctgagatggcgccacttgagtttcaagtcaaggctaagaaaaaataattaattaaaaataaaaacaagaagatgccactgcattccagcctgggtaacagatgctatctcttaaaaataaaaaaatcataagtttcctaggcttagagagaaccttgggcaccctctgtcttcaggcaggccagtgcctatttatcgattccacgcgtgtttacggagcacctactatgtgccaggcactgtcctaggcaacacaaatacaacagttaacaaaacagagtccctgtcctcttcacagcctgccagagcaaacatgccccacaaagttgtccccacaaatatcgaattcatcatcattatgatgataaaaaataaaacaactcagccgggcaccatggctcatacctgtaatcccagcattttgagaggctgaggtgggcagattactcgaggtcaggagtttgagaccagtctggccaacatggtgaaacctcaactctactaaaaatacaggcaatgcctgtaatctcagctacttaggaggctgaggcacgagaattgcttgaccccgagaggcagaggttatagtgagctgagattgcatcactgcactccagcctgggcaacagaacaagatcctgtctcaaaaattaaaaaataaataaataaaaccactcttcaacaaatgcttatgatatgctaggcactgtgttcagtgttctttggataattggtgtcattctaatccacacaaccaccctaggattgggaactcctgtcgcagccccagtttacaggtgaggagatggaggttcagagaagtaagtgagcttgcctggagatgcacagtggaagactgtgggatttgaatctaggcctgatgtgatcccagaactgctactttttaaccattcttccggattgcccgttctccattctccgcttgatagcaagacctgtgaaggggacacaagaggagacccaacctagtcagggttatggagagacagggagtcagtccaaaaacaaaaaataaaaaaaagtgccctaaggaatgagtagaagttggccagatatggtgaagagagtgcccccagtcttggatacagcaggtgcagctggtgagatgcttggtgcattcaaggaactgaaagaagggctgagtcacagaaaacatttttatccaaaagagagtcctgaccgggcacagtggctcacgcctgtaatctcagcattttgagaggccaaagcgagtggattacttgaggtcaggagttcgagaccagcctgcccaacatggtgaaaccccatctctactaaaaatacaaaaattagccgggtgtgctggcacatgcctgtaaacccagctactcaggaggctgaggcaggaaaatcacttgaactcaggaggtggaggttgcagtgagccgagatctcgccactgcactccagcctgggtgacagagcaagaccttgtctcaattaaaaaaaagaaaagaaagaaagcaaaagaaagtcctgactgggcacgggcacagtggctcaggcttgaaatcagagcatgttgggaggccgaggcttaaggatcacttgaagccaggagttcaaggtcagcatggccaacatggtgagacagcatctctgtttttaaaagtcctagaagggaattctcacatagtgattactttcccaattatcagaaagtcccttctttctattaatagggttcttcctgccttggtgggttcttagagctgctcaagagaatctttaataataataggaacgacagcactccttctgggcactgatccatgcattttataagcagcacctcattgaatcttcccaacgattctctgggttacttatttttatccccctgttttgtagatgagagaacagagcctcagggaggttaagttgcccaagatattaagtggcatgccaggattcaaacctggagcccatacagtgacccatcaagcttcccagaagctgtgaacatctaggctgtaataacaccaccacctgcacagacgccacccttccccatcctcctgttcctgtgtacacagatttaatggtcagggaactggcatcgaaggccaacgttagctctggctgctgtacacctttgtaccaggaccttggcctctgtccagcatttaatgctcctgcaggaagttctccagccagagttggcatttgtggcctcaattaccccactttgaggcagggtcttgggtaaattggacatgaagtgactggggtgcagggcccggccccgcagagatacttcttgatagatccatctctcactggccaccatgtccaacatgggagcgccacagggctgagggaagacattggagccagcctcacagtcccattcattttctctctcttgaaactggctcctgttcccaaagttgctcccccctgggagggagatctcagaacccccataaacatacccactggagcccaccaaacccagattctaaactcacctctgccctttatcagtggagtgagcttgcaaaatcacaccaactcctggagcctcagtttcctgagctatatatggggacggtaacagacttgtggcaaacaagacctggaaagcacctagcacctggcacatggtaagctatcaataaccatgagccattacaattcttcctcactcgaatgtgaatttcataaggaaagagaccaggtccctcttgtttgctactgtatcgtctgtgccttgcacagtgcctggcacatagtaggtgctcattaaaagttggtgaatgaatgagtgcatgggtggtggaaagatggatgcatgggtgggtggacggagaggtgggtgcttaggtggggtgttcttcttatacatagtggcagccatgctggagtagcagtgtgatatggttggctcagtgtcaccacccaaatatcatattgaattgtaacccccatgtgtcgagggagggacctggtgtgggtgattagatcatacgggcggctttccgcatgctgtgttcgtgatagtgagtgagttctcacaagatctgatggttttaaagtggcagtctcccctgtgcactctctttctctctctctcctgctgccatgtaagacatgccttgcttcccccttcaccttccgccatgattgtaagtttcctgagacctctctccagccatgtagaactgtgagtcaattatacctcttctgtttataaattacccagtctcaggtagtatctttatagcagtgtgaaaacagactactatggagtgggtcccagatttcatttgctctgctttacagatatgcattccttccccctaaccagccctctccccatccaggtattccaaaatcaagcatt

SEQ ID NO: 2 - DNA, 12362 bp, Homo sapiens
cctctataggaatgcacctggtaagagaatattctcatttccataggcttggagagaacagatctttttttttccaaggataaccaatggggtgcagcttatggaaaagggtgtttgtttcaaaagggaacatggtcccagattttcccatggttatcccattcatttcaggagttacattaaaggttgtgtttgtatcttctggccggctgcagtggctcacacctgtaatcctagcacttcaggagaccgaggtgaggtcaagagtttgagaccagcctagccaacttggtgaaacctgtctctactaaaaatacaaaaaattagttgggtatggtagtgtgtgcctgtaataccagttactagggaggctgaggcaagagaatagcttgaacccaggaggaggaggttgcagtgggccgagatcacgccactgcactccagccgagcaacagagcaagactccatctcaaaaaaaaaaaaaggttgtatttgtatggattaataggacagggtgccatttggggagagaatttcaagacaccagcatttatggaacctatgctggaagcaaggcattaggacactaactatatatttgtcagtaatacaaacagataagattaccgtctatctttctgggctgcttagaggcctatgaggcatgtcaataactgtattaaagcctagtggaaagtcactcagctatccaaagtctatttagcccattacactcctgtcaattttacctcatttaccaagaaccccttctccctccaaatttgacctttgtggacctcattgggcactgagaacatgcaaacctatcaacagaagcacaagtaagccgcagactccaatccccgagtcagccttgcccaatgggagctgaatggcaagatgaagagtggactctgatggccttggtcactccactgcttatggagttttcttgtttgcaggcctggggtctttttattccagtatctatctgcctctccctttctctctttcatagaatgactgttaagaacttatttagaccgggcacagtggctcatgcctataatcccagcactttggaaggccaaggtgggtggatcacttgagcccagaagtttgagaccagcctgggcaacatagcaaaaccccatctctaccaaaaacgaaatacaaaaaaaatcaccaggcatggtggcacaggcctgtagtcccagctactcaggaggctgaggtaggaggatcacctgacctcagggaggtcgaggcttcagtgagccatgattgtaccattgcactccagtctcggtgacagagtaagactctgtctcaaaaacaaacaaacaaacaaaaactgatttaaaggagttaggtgcagtgcatagacttgtagtcccagccactcaggaggctgaagtgggacgatcacttgagctcaggagttcaagaccagcctgggcaacatagcaagaccccatctctacaaaaaatataaaattagccaggcatgctggggcacacctgtagtcccagctttgcaggaggctgagtggggaggagtgcttgagcccaggagttcaagacccagcttgggcaacatagtgagaccctgcctctattttttttttaagaacttatttaaagagagtgtccacaccaaccataccaaggagtgtaccaagcagagtgtatctctcttccactcccgacatccagacttctccttcctctccagagggaccaccactgttgctactgtgtcttggcagggatattctagtcattcaagcaagtgcatataggtataagcatttctccttttatgcaaatacaaaatatttctcacttaatgctatagctgggaaatggttccatatcaaaacatatagatctgcatcattcttttaaaagattcatagtcttccattgcgtgaataagttattgatgcaaccccccattgatagacattaggtcatttcaggcacaactaaaagcaatggtgcaatgtgacgtgtatttgcccagttacttcctagaagtttgtcaaacaggacatgtgtttgccagattgtcccccaatgaggatatagcaattgagaaccacaccagcaatatgagagtagctatttccccacactctaaccaacataatgtattaatgtttaatctttgctaatctgtcaagagaaaaatggtatctgttatcattttaatttcattctttcataatgatgtttttcttttcttttcttttcttttttttttttttttttttttttttgagacagagtgtcactctgtcacccaggctggagtgcagtggcatgatcttggcttactgcaagctccacatcccaggttcatgccactctcctgcctcagcctcccgagtagctgggactacagtcacccgccaccacgcctggctaattttttgtatttttagtagagacagggtttcactgtgttagccaggatggtctcgatctcctgaccttgtgatccgcccgcctcagcctcccgaagtgctgggattacaggcgtgagccacagcgcctggcccataatgacgtttttctttacctgtttgtgggtggggagaaagaaacaaaatctccttgtggactatacatttgttcatgcctttatttaaatccaaccttaatatccccaattaaacttcctagcttttatccttcacaaacatatcctccagagaagtggtttccaaacctggttgattatcagactcaaagccactgaatcagaatctctagaagatgagcccaaggatctatatttttaccaagttgtagtggttattctaagaggcagcacccttgggagtcttggcttgacctttttttaatgtttaaatttaaaaaatttttagagtcaggttcttgctctgtcacccaggctggagtgcagtgacacaatcatagctcactggagccttgaactcctgagagccttggctttagagcaatgcagaattagaggaaaagctaattgtgtcttcccaagtaaaagtgaagctagaatgacacaaagttctagctatagctttgcattgggttacataaaaagatggtgggcaattagggcttggatatccgcctgtcatttatccattcaattgacattttctgagcaccttctatgtgctagacatggggcacctcagtgtggaggacaccgtccctgtcctgagagagtctagagtccagcagagaggattgagtaataaaccagaaataatacaaatgattcattataattgccataactgctatgaagaagaaatgtaagaccatatgagtgcatattatgacacgactgacctggggctcattttcaatggggaagatgaggttcaaggaaggctttcctgaggaggtgctatttaagctgagacagcgagatgatgaaccggaatggaagtatgacagatgaaatctggggcttcactgcctcatggtgccccatgaaaaggacaacactggacaagggaaaccatcaaaatggtctttctgggcccccagatgctctcaggaggcatgttacttaggggctttgttccagcatgggagggaagtaatgtgtatcagcttgagctgtgtgaatgaaaaaggaaaaactatctcgaaccctcaatctgctcaaactgtgacttgaaaagacttttttaagagatagccacttaagcagctgggcaactcattcttttgttcactggcagatggcaaaacctcagagcacaatggtgagcattttggtcagacatgtcccacacacttttatctgcaccctgaagtaggatccaggatttgcagcgcatccttcccttatcctttcgccctcttcccccttttcaaactggtgtaagttactagagcttgtaaaagtataagaactaaggaattctgctctcttgttttcttgctctgagcttggaaatacccaccacattgttaagcaaatgttctctttaccagtagtgggtaaacaaagacatgagtgagttttctgctacacctaatttaagatgtagaaaatcaaagccacagagcagggtcagtcagctctaggtctgatttatgagcatttagaacaacaacaaaaaaaaaaacagggtttatgaagaggtcagcagagtgtcactaagaaaaactccaaccaaactagacttccttcctttcttcttttctgattttatttatgtattgattgattaataggtgtgtgaagctggtaggctcagtcaggctggccaggcagttggtaaacaaaacttcctttattcactgagagattgtgtgtccaaagcactgaatattaaaaggtgaacccaacagagctcctaccttcaaagttcctaactttcagaggggaagacaaaagtttgtttgcttttttaaagacaaggtcttgctctgtcatccagctggagtgcagtggtgtgatgacagctcgcagatcacagcaacctcgaccacctggggtcatgggattctcctgcctcagccttccaagtagctgggaccataggcacatgccaccatatctggctaatttttacatttttgtagagatgggggtctcgctatgttgctcagactggtctcaaacccctggcctcaaacagtcctcccatcttggcttcccaaagtgctgggattatagatgtgagccaccacacctgaccaaagacaaaagtaagcacaactttgcaatacagtttccatctctgtggaaacagaccttttctgcctggttccaaagcttacttcttagaacataacagatcctcagtaaatattgatagatgacagatggatgggtgggtgggtgggtggatggatggatggatggatggatggatggatggatggatggatggataggtgagtgggtagatgggtaggtggatggatggctggctggctgaatggatagataggtgagtgggtgcgtgggtaggtgcatggatggatggatgcccagacagaagctactggagaaggctttctaaataatggatttagaaggacaagtgcaggcaaatagtcaagaagagccagtccagacaaagaatgtagcaattacagaggagtcacaaaaaggagtttatttaagctgtgtagagaaacttttcaaaaaatcattttagtggaagtacaatcagtacactggagacagcacatttgtagtaattctagaaagaaatgatgtggacctcagctgaggcaatggctgtggagatgagcagaggaaatagatttcacggatattatagcacagaagcaacaggacttgacagccaatgttagccagaagggccgggggaagttagattgatatttaggtcctggcttgggcagggagatggaggaatgccaggctcagaattagagaatccaagggcagggtatgtttttgggagaagatgaccacttcgatttgggacagacccaatttgttatgcctttgagaagtcaaggtgaaaagaaagcattcattagatgctatggacctggagttaaggagaaaaccttggggggtaattgtagacatggaaataaatgccatcacccaagacctgagacagaaccctaaacatgcaaatgtttaagggctgagcagaaagtaaaaggagaaagagaggcagataagtaggaacagaagccagaaagagttggcatcagataaatcaaggaagagaattttacagaaggtgtgaccaaataagcaagtacagtcgagaggtcaggcaaggtagaaggcaaaagtattctctggttttggcagccaagaggtcaccagtgttctatgccagagttgttttcagtggaaggagtggaacagaaaccgaagtgcagtgggtcgaatggtgaatggaaaatgaagaagtggacgtgggtggaccactcacccagtggggttggggaccaggccctggagaatggggagactccatgtcattacaaatcctgttgtacattttgattctaaagcacatagccagactttatttcacaagttcagtctatgccaaacagggattaagcgctctgatgggtcaggcgcggtggctcacacctgtaatcccagcactttgggaggctgaggtgggcagatcacgaggtcaggagattgagaccatcctggctaatacagtgaaaccctgtctctactaaaaatacaaaaaattagccgggtgtggtggcaggcgcctgtagtcccagctactcgggaggctgagacaggagaatggcatgaacccgggaggtggagcttgcggtgagccgagatcatgccactgcactccagcctgggcgacagagtgagactccgtctcaaaaaaaaaaaaaaaaaaaaaaaaaaagagctctgatggctgagattcaaacccaccccagttttgcacattggttgtataatctctgcctcagtgatctcatctgtaaaatagggatgataataatgacgttacttcatggctggtgagaatttaatgagttggtgcatgtgaaacgcttggaaatgtgtttggcatatagtcagtgcttaaaatatattggcttggctgagggcagtgacttacacctgtaattccagcactttgggaagttgaggccagtggatcacttgcacctaggagatttgagaccagtctgggcaatatggtgacaccccatctctacaaaaaatataaaaattagccagccgtggtggcacacacctgtagtcccagctacttgggaggctcatgcgggaggatcacttgaacccaagaggcagagattgcaatgagctgtggccatgccactgcactgcaggctgggtggcagagtgatactccatcacaaatatatacatatatatatatatatatatatatatatatatatatatatatatatagagagagagagagagagagagagagagagagagagagactattactatatattttttaagaaaagaggtgaagagaaggaaaaataactttgagtgaaaaaggaaaaagaggggttagggaaggtttctattgtcttttttgacacctctctcctttatgaattttatatatatatacatatataatatatatttatattatacactactagtacattatatataaaagatactattaataattatacatacataaaggataatgattatttgccgtatgtgttgcaaacatctttcccagtttgtggttttctttagggtactttttcacatgcaaaatagtgaatgtttacatagtcaaagtctttgttttcttctttaatcattatgttaatgagagttcttcctctttgagaccagatactaaattgagtttgcttccagttattttattatttcatgtaatattctaaaccatcagaatttttttcaccgggtacagtggctcacgcctataatcctagtgctttgggaggccaaagtgggagcttactgccgctctgacctcctgggttcaagcaatcctcccacctcagcctctcagatagctgggattataggcgtgcaccaccacacctacctaaatattgtatttttagtagagacggggtttcgccatgttagacaggctggtctcaaactcctgacctcaagtgacccacccgcctcagcctcccaaagtgctgggattacaagcatgagctatcatgcctggtctattgttgttattgtttaatcaaaatcaaatagccactaggtagcacagtgggaatctgatttcagacttgttcggatccaagcctacattcctgaccccttagctagcctgcctggtgctcccggttgtttaggggtgaatgaccattgaccataatacggtccgttgagaaaaagccttctagaagggcccagaattcaacccccttacgcccacggtcagaggagaccatcaggacagggtcatgggaggggccctgagacagcctagagacccaaagcaaacaaaacctcctcaataatgagtccaagctgggcacagtggctcatgcctgtaatctttgggaggccaaggagggcagatcacctgaggtcaggagttcaagaccagcctgaccaacatggtgaaaccccatctctactaaaaatacaaagaaattagccaggcatggaggcacacacctataatcccagctgcttgagaggctaaggcatgagaatggcttacacccgggaggtggaggttgcagtgagctgagatcatgccactgcactccagcctgggcaacagagtgaggctccatctccaagaaaaaaaaaaaaaaagtccctcccaggtctgcacaggttcaagaagaaaaccatccacctgttccatccccagcctggccatccctgctgtcccctccctggcagaagcagcagcagtccattctgagaaacaggggtatagcatggaaaccaagggaaagaaaagccccccacccacccccagagcccagctgccactttagcctgttgactggggctggtagacacctccaccatgccctcagtggctgggggaggaagtcctttttactactgggagaatctctcactcaaacaaagtcttacccagaaatgtacacaatggaaacagaaatgcaggatgaatgtagctgagcctgggaaaccagcctgcttaccgcccaccccgctgtgatgggccctaaaacagggtgcagacagaacagaatttgcaaacctctaagctgtggcaagacagcaatctttctgagaaggtgacttgggattactggctgggacaagagttaccactgaaatgtgagaacaatggtctgatagccaccttgtctgggaatagcttggctggactgaccaccttacagatgagaagtgattatactggttggaaggtctcaaggttgcaagaaggtgggagctggctctctaaccccagaagctgtgggggctttgataaaggccctcctcttgtcacctcccttcccctcaggctctgctgccatctgggctttcccccttagcactgtttataccttaaagaatgagcttgctgggtgcagcgccccatacctataatcctagcactttgggaggctgaggtgggaggattacctgagcccaggaggtcaaggctgccatgagccatgattgcaccattgcactccagcctgggtgacagatcgagaccctgtctcagagagagagggtggtgagctaaggactcctatacaattgtctccttcccccaagatcttcagaaagaagcagaaagaatctggtttgatataaatgggctccgtgtcccacacatatacccctatacctacacatacatgcccacatccactcacacacatccacacatacctatgtgtgcacacacatacacacttttacatgcataagtgtatgcatagatacacatacaaactcacacatatgcacacatatacacactcttttttgtgtgtccctaggggtcaggatagaagtcaaaatgtcttcctagcctccacaagccagcagttctagaaaactggaagctggggccgagggaagagcattctgtcaaagttcatgcattcgaggcatgaacagaaaatttgctccatttgctctacttctggttgcttcggaaaacaacctgaacctttcaagagctgtgagtttgataagcatcgttgcagaatgaagggccattgagtactcagaaagcgttgaaatccaaatggtttctcataatcagtttcctggaagcaggccatgggctggttttcagggtagagggagccccagccacccctgctggctgcactcggcttgcctaagtggacagacccctcatctggatgccagagtattatgctttcctaccccaggaagctagagcctgtagctgacactctagactctgggaacagttcagcaggaccggggggaccaaaggaacgcttctgcccagggatcctgctgcttggagaagcttagaggatggcaaggaattgtgacctcaaggaaggccactgcttggcatctttgcacctctgtgcccagcccagccctggtgtgcactagaggccctgcatgcatttgctacccatctattaagtctatttattctgtactgacatagaaagacactcactcttgttagtgagaagggcgggttgcagggcaaagtaaatgtatgagtccatgtctgtctgtctgagatgcaagatctgatctgctagtgcgggcaccctgggaaagaggggaggccagacagccactttcccctttgtttgcttctgttgtttaaacagttttaaatgagcatcgtcaattgccctggaggtacttgcagctaattgagggagactaatgggtcagcagatgaaatgcaatacaaacagaaagggattataccagttgagatggctagtttccacctgcctcaaacctcttaattccttccccctctacccgttttcattctttcttcaaagagaaaataattttcttccctcccacaaatctgccattctttttgtctttacccccatctccccaaccaaaaaaataaaaacaattatacatgggcacgtggatgcacacagcacacaaacacacagttgtaataaactggtcactatttatcttccattgagttgctgaccttagaagatgttatcctcccacccccatttttgcttgtttggcttggctggattatttatttgcagtgacaaatattaagctagagcaaaatttagtaatccacaataaaacatcattgaagccaggtgcagtggcaggagcctgtggtcccagctactcagagaggctgaggcaggaaggtccattgagcccgggaggtagaggctgtagtgacccttgattgtgccactgcactccaccctgggtgacagagtgagaccctcatctctaaaaaaaaaaagcaaaaaaaattattgaataaggattttgaattttgagtgacaatgctgtttacctcctctcatgatcagggtagcattctactctgttgagctttccttagcttttttctaagaaagtcaacatccttcaagtcaacttctgggaccagaaaacaaggcaatgggtccttcacctgagcgagaatcccaagcactcacagtccctgctcccgcttcctttctctcccttcccttcccttcccttcccctcccctcccctccccttcccttcccgcccttccttcctttctctctctctctttctttctctctcttctttcttttcttttttccttcctttcttccttccttccctccttccccccttccctccctctctctctctctttgttttcaaaatagggtcttcctctgtcaccaacgctggattgcagtggcacaaccatagctcattgcagcctcaaaccctggactcaaggaatcctccctcctcagtctcccaaaatgctgggattacatgcatgagccactgagcctggcccctgctcatctttctaatccctcctcccacgtcccagtggatttgccaactgcaaattcatttcattcagacaatcaaatccaaatttctcagctgggcatgatttggcctcaacctccccatctgactttatctccagcaatttctctattttgccattttcagccataccagcccatcttgcttcaagcctttcctgtttgctctacccaatcgtactctgtccacttcatcctcttttttaaagatagagtctcactctgttgcccaggctggagtgcagtggtacgacgatggctcactgcagccttgacctcctgggctcaagtgatccccccatctcagcctccctagtagctgtgactacaggtgtgtgtcaccattcccgacttcctgtccacttcttgacaagaccagctctttcttgcagttccacgctaagccaacaggtccctttttcggaaataccttccctaatcactctatgggaaaaactgcccatctgctcttaacgcctccagtttccctaccccatttcattaaacaccatccagatttatccaaaatatcttgtttatttatttgcttgctgaccatttgtttaccccactggaaggtacgttccaggagagcagggaacttatctatcttgatcattgctgatttcagtgtctaccacagtaccaggcacctaataggttctcactaacttttcattaaatgaatgaccaaatagcttctcagctcccaagggctgcttgcaaagtcccctgtctggtgctccttgcaaagcccccgcctgggtccggggggaggacagggctgagtgtgtgctgctgtgattgcagacagagtccttcaagctgag

SEQ ID NO: 3 - DNA, 20 bp, Artificial Sequence
  PCR primer SCNN1B forward
acccagtctcaggtagtatc

SEQ ID NO: 4 - DNA, 19 bp, Artificial Sequence
  PCR primer SCNN1B reverse
ccagcgagactcaaattac

SEQ ID NO: 5 - DNA, 21 bp, Artificial Sequence
  PCR primer SCNN1B forward
gagtgggtcccagatttcatt

SEQ ID NO: 6 - DNA, 23 bp, Artificial Sequence
  PCR primer SCNN1B reverse
ggtccttcagcaaatgcttgatt

SEQ ID NO: 7 - DNA, 18 bp, Artificial Sequence
  SCNN1B probe
tgctttacagctatgcat

SEQ ID NO: 8 - DNA, 19 bp, Artificial Sequence
  SCNN1B probe
ctgctttacagatatgcat

(19) United States
(12) Patent Application Publication
(10) Pub. No.: US 2009/0149428 A1
(43) Pub. Date: Jun. 11, 2009

(54) Methods for Assessing the Predisposition or Susceptibility to COPD

(75) Inventor: Simon Smith, Cheshire (GB)

Correspondence Address: FISH & RICHARDSON P.C., P.O BOX 1022, MINNEAPOLIS, MN, 55440-1022, US

(73) Assignee: AstraZeneca AB, Sodertalje (SE)

(21) Appl. No.: 11/719,607

(22) PCT Filed: Nov. 16, 2005
(86) PCT No.: PCT/SE05/01726

§ 371 Date: May 17, 2007

(30) Foreign Application Priority Data
Nov. 18, 2004 (SE) 0402832-0

(51) Int. Cl. A61K 31/522; C12Q 1/68; A61K 31/197; A61P 11/00; A61K 31/56; C07H 21/00

(52) U.S. Cl. 514/171; 435/6; 536/24.33; 536/24.31; 514/263.34; 514/562

(57) ABSTRACT

This invention relates to novel associations between polymorphisms in the genes encoding the human Epithelial Na+ Channel (ENaC) and COPD. More particularly, the invention relates to a method for assessing the predisposition and/or susceptibility of an individual to COPD, which method comprises detecting the presence of a polymorphism in one or more human ENaC-encoding genes, in particular position 3870 of SEQ ID:1 encoding SCNN1B or position 10544 of SEQ ID NO: 2 encoding SCNN1G. The invention also relates to a method of treating a human identified as having a polymorphism in one or more human ENaC-encoding genes with a drug capable of treating COPD.

This invention relates to polymorphisms in the genes encoding the human Epithelial Na+Channel (ENaC). The invention also relates to the use of polymorphisms in the ENaC-encoding genes in assessing predisposition and/or susceptibility of an individual to chronic obstructive pulmonary disease (COPD). The invention also relates to the use of polymorphisms in the ENaC-encoding genes in the treatment of diseases with a drug capable of interacting with ENaC or one of its subunits.

The pathophysiology of COPD is complex and poorly understood. Current clinical guidelines define COPD as a disease state characterized by airflow limitation that is not fully reversible. The airflow limitation is usually both progressive and associated with an abnormal inflammatory response of the lungs to noxious particles and gases. The most important contributory source of such particles and gases, at least in the western world, is is tobacco smoke. COPD patients have a variety of symptoms, including cough, shortness of breath, and excessive production of sputum; such symptoms arise from dysfunction of a number of cellular compartments, including neutrophils, macrophages, and epithelial cells.

Mucus hypersecretion, thickening of the mucus and impaired mucociliary clearance (MCC) are well-established features of COPD pathophysiology, and contribute significantly to the morbidity and mortality of the disease. Small airways can become occluded by mucus plugs, leading to severe airway obstruction. Additionally, excessive mucus production in large airways can result in symptoms of chronic bronchitis. Impaired MCC also gives rise to increased rates and severity of exacerbations.

ENaC is a non-voltage gated channel that mediates transport of sodium ions across epithelia and is considered rate-limiting for sodium transport in many tissues including the lung, kidney and colon. In the lung, regulated ENaC activity is essential for the maintenance of air surface liquid (ASL) volume.

ENaC channels are heteromultimeric proteins composed of three homologous subunits, α, β, and γ; however, the subunit composition and the relative contribution of each subunit to channel function is not entirely clear. One frequently cited model suggests that ENaC is a tetrameric channel with a stoichiometry of α₂β₁γ₁ (Firsov et al., EMBO J., 1998. 17(2):344-52; Kosari et al, J. Biol. Chem., 1998. 273(22): 13469-74).

A role for ENaC as a key regulator of ASL volume and mucociliary clearance (MCC) in vivo is established in the literature. Studies on α- β- and γ-ENaC (−/−) mice, respectively, show a critical role of ENaC function in perinatal lung liquid clearance (Barker et al., J. Clin. Invest, 1998. 102(8):1634-4G.; Bonny and Hummler, Kidney Int., 2000. 57(4):1313-8.; Pradervand et al., Proc. Natl. Acad. Sci. USA., 1999. 96(4): 1732-7.).

Furthermore, humans with Pseudohypoaldosteronism 1 (PHA1, loss-of-function mutations in the genes encoding α- β- and γ-ENaC) show increased ASL volume and an upregulation in MCC (Kerem et al, N. Engl. Med., 1999. 341:156-162) and treatment of normal subjects with the ENaC channel blocking compound amiloride increases ASL volume and MCC rates (Sood et al, Am. J. Crit. Care Med., 2003.167:158-163.). The role of ENaC in cystic fibrosis (CF) has been extensively investigated. For instance, Matsui et al. (Cell, 1998. 95(7): 1005-15) showed that airway epithelia of CF patients exhibit increased rates of ASL absorption, depletion of periciliary liquid (PCL) and decrease in MCC rates.

As mentioned, the pathogenesis of COPD is ill understood. Several factors, such as inflammation in the peripheral airways, are thought to play an important role. However, these factors do not explain why, for example, some smokers develop COPD and others do not. Accordingly, we hypothesised that other factors, such as unidentified genetic factors, may be involved in the pathogenesis of COPD.

The present invention relates to the surprising discovery of an association between polymorphisms in the human ENaC encoding genes and COPD. The alpha, beta and gamma subunits of human ENaC are encoded by the SCNN1A, SCNN1B and SCNN1G genes, respectively. Accordingly, the term “ENaC encoding genes” refers to human SCNN1A, SCNN1B and SCNN1G genes.

SCNN1B (also known under the names ENaCb, SCNEB, ENaCbeta, beta hENaC) can be found in NCBI Reference Sequence project (RefSeq) under Description: sodium channel, nonvoltage-gated 1, beta (Homo sapiens); Accession number NP_{—}000327. SCNN1B is present on chromosome 16 [Location (based on Ensembl build 34): 23280185-23359172Strand: +]. The gene structure of SCNN1B has been described by Saxena et al, Biochem. and Biophys. Res. Comm. 252, 208-213 (1998).

SCNN1G (also known under the names SCNN1C, PHA1, ENaCg, SCNEG, ENaCgamma, gamma hENaC) can be found in NCBI Reference Sequence project (RefSeq) under Description: sodium channel, nonvoltage-gated 1, gamma (Homo sapiens); Accession number NP_{—}001030. SCNN1G is present on chromosome 16 (Location: 23160591-23194756 Strand: +). The gene structure of SCNN1G has been described by Thomas et al, J. Biol. Chem. Vol. 271, 26062-26066 (1996).

SCNN1A (also known under the names NaCh, and alpha hENaC) can be found in NCBI Reference Sequence project (RefSeq) under Description: sodium channel, nonvoltage-gated, type I, alpha polypeptide, sodium channel, nonvoltage-gated 1, alpha (Homo sapiens); Accession number NP_{—}001038. (See also Voilley et al., Proc. Natl. Acad. Sci. USA, Vol. 91 (1), p 247-251 (1994)).

Polymorphisms can help identify patients most suited to therapy with particular pharmaceutical agents (this is often termed “pharmacogenetics”). Pharmacogenetics can also be used in pharmaceutical research to assist the drug selection process. Polymorphisms are used in mapping the human genome and may be used to elucidate the genetic component of diseases. The reader is directed to the following references for background details on pharmacogenetics and other uses of polymorphism detection: Linder et al. (1997), Clinical Chemistry, 43, 254; Marshall (1997), Nature Biotechnology, 15, 1249; International Patent Application WO 97/40462, Spectra Biomedical; and Schafer et al. (1998), Nature Biotechnology, 16, 33.

Clinical trials have shown that patient response to treatment with pharmaceuticals is often heterogeneous. Thus, there is a need for improved approaches to pharmaceutical agent design and therapy.

As mentioned above, the present invention relates to the surprising discovery of an association between polymorphisms in the human ENaC encoding genes and COPD.

According to one aspect of the present invention there is provided a method for assessing the predisposition and/or susceptibility of an individual to COPD, which method comprises detecting the presence of a polymorphism in one or more ENaC-encoding genes.

The term “ENaC-encoding genes” refers to human SCNN1A, SCNN1B or SCNN1G genes.

The term polymorphism refers to a sequence variation observed in an individual at a polymorphic site, and includes single nucleotide substitution, nucleotide insertion and nucleotide deletion which in the case of insertion and deletion includes insertion, or deletion of one or more nucleotides at a position of a gene. Preferably, the polymorphism is a single nucleotide polymorphism.

Preferably, the one or more ENaC-encoding genes are selected from human SCNN1B or human SCNN1G.

In one embodiment, the method comprises detecting the presence of a polymorphism in human SCNN1B.

In a further embodiment, the method comprises detecting the presence of a polymorphism inhuman SCNN1G.

A particularly strong association has been found between COPD and a single nucleotide polymorphism in intron 2 of human SCNN1B. This single nucleotide polymorphism is defined as that corresponding to position 3870 of SEQ ID NO:1. SEQ ID NO:1 is the first sequence listed in the attached sequence listing. SEQ ID NO: 1 shows the sequence of intron 2 and is spanned on either side by the last 20 nucleotides of exon 2 (positions 1 to 20) and the first 20 nucleotides of exon 3 (positions 3911 to 3930).

Accordingly, the present invention also provides a method for assessing the predisposition and/or susceptibility of an individual to COPD, which method comprises determining the nucleotide of the individual at position 3870 of SEQ ED NO: 1.

It should be noted that whilst SEQ ID NO:1 in the attached sequence listing shows base A at position 3870, the method encompasses determination of the nucleotide corresponding to position 3870 of SEQ ID NO:1 in the individual being assessed, whether this comprises is base A or otherwise.

In a further embodiment, the method comprises detecting for the presence or absence of A and/or C at position 3870 of SEQ ID NO: 1.

In a further embodiment, the method comprises detecting for the presence or absence of A at position 3870 of SEQ ID NO: 1.

A further association has been found between COPD and a single nucleotide polymorphism in intron 6 of human SCNN1G. This single nucleotide polymorphism is defined as that corresponding to position 10544 of SEQ ID NO:2. SEQ ID NO:2 is the second sequence listed in the attached sequence listing. SEQ ID NO:2 shows the sequence of intron 6 and is spanned on either side by the last 20 nucleotides of exon 6 (positions 1 to 20) and the first 20 nucleotides of exon 7 (positions 12343 to 12362).

Accordingly, the present invention also provides a method for assessing the predisposition and/or susceptibility of an individual to COPD, which method comprises determining the nucleotide of the individual at position 10544 of SEQ ID NO:2.

It should be noted that whilst SEQ ID NO:2 in the attached sequence listing shows base G at position 10544, the method encompasses determination of the nucleotide corresponding to position 10544 of SEQ ID NO:2 in the individual being assessed, whether this comprises base G or otherwise.

In a further embodiment, the method comprises detecting for the presence or absence of C and/or G at position 10544 of SEQ ID NO:2.

In a further embodiment, the method comprises detecting for the presence or absence of G at position 10544 of SEQ ID NO:2.

It should be noted that in this application, SNPs are referred to by reference to a position in SEQ ID NO:1 (e.g. position 3870) or SEQ ID NO:2 (e.g. position 10544). However, when such references are made, it will be understood that the invention is not to be limited to the exact sequence as set out in that listing but includes variants and derivatives thereof. Instead, identification of SNP locations in similar sequences are contemplated (i.e. SNPs at positions which the skilled person would consider correspond to the positions identified in the SEQ ID numbers). The person skilled in the art can readily align similar sequences and locate the same SNP locations. The position of the SNPs refers to the position in SEQ ID NO:1 or SEQ ID NO:2 where the first nucleotide in the sequence listed is position 1.

It should further be noted that detection of the nucleotide in the complement strand to SEQ ID NO:1 that base-pairs with the nucleotide at position 3870 of SEQ ID NO:1 is of course within the scope of the claimed invention. The same applies to SEQ ID NO:2, i.e., detection of the nucleotide in the complement strand to SEQ ID NO:2 that base-pairs with the nucleotide at position 10544 of SEQ ID NO:2 is also within the scope of the claimed invention.

The term individual means a human, and includes a human having or suspected of having COPD and an asymptomatic human who may be tested for predisposition or susceptibility to such a disease.

In a further embodiment of the invention, the present invention provides a method for assessing the predisposition and/or susceptibility of an individual to COPD, which method comprises determining the nucleotide sequence of the individual at position 3870 of SEQ ID NO: 1 and position 10544 of SEQ ID NO:2.

In the context of the present invention, detecting the presence of a polymorphism in one or more human ENaC-encoding genes means determining the identity of one or more nucleotides at a polymorphic site in an ENaC-encoding gene of the individual.

The polymorphic site will be one which has an association with COPD in a human population. By this is meant that a particular nucleotide or nucleotide sequence at the polymorphic site is correlated with incidence of COPD, and occurs at a greater frequency in susceptible patients (for example a site which contains e.g. a single nucleotide substitution, nucleotide insertion and nucleotide deletion which occurs with greater frequency in COPD patients/COPD susceptible subjects). Methods of identifying such sites in the ENaC-encoding genes are described herein with reference to SNPs. However, the same principles can be used for other polymorphisms. In this way, the individual can be genotyped with respect to the particular polymorphic site. The polymorphic site may correspond to a polymorphism selected from a single nucleotide substitution, nucleotide insertion and nucleotide deletion which in the case of insertion and deletion includes insertion or deletion of one or more nucleotides at a position of a gene. Preferably; the polymorphism is a single nucleotide polymorphism. The method of the present invention may involve determining the identity of one or more nucleotides at two or more polymorphic sites in one or more of the ENaC-encoding genes.

Methods for determining the sequences of nucleic acid sequences and determining the identity of nucleotides at particular positions within a sequence will be recognised to those skilled in the art and suitable methods are described herein.

The method for detecting the presence of a polymorphism in an ENaC-encoding gene may, for example, be determined by a method selected from amplification refractory mutation system, sequencing, allelic discrimination assay, hybridisation, restriction fragment length polymorphism, oligonucleotide ligation assay, or allele specific PCR.

The test sample of nucleic acid is conveniently a sample of blood, mouth swab, biopsy, or other body fluid or tissue obtained from an individual. It will be appreciated that the test sample may equally be a nucleic acid sequence corresponding to the sequence in the test sample, that is to say that all or a part of the region in the sample nucleic acid may firstly be amplified using any convenient technique e.g. PCR, before analysis of allelic variation. In one embodiment of the invention, the detection of the polymorphism is determined from a nucleic acid sample (which may be as defined above) that has already been removed from the individual. Therefore, in each aspect of the invention where the analysis of nucleic acid is required, the invention includes the case where the nucleic acid sample has already been removed from the individual.

It will be apparent to the person skilled in the art that there are a large number of analytical procedures which may be used to detect the presence or absence of variant nucleotides at one or more polymorphic positions of the invention. In general, the detection of allelic variation requires a mutation discrimination technique, optionally an amplification reaction and optionally a signal generation system. Table 1 lists a number of mutation detection techniques, some based on the PCR. These may be used in combination with a number of signal generation systems, a selection of which is listed in Table 2. Further amplification techniques are listed in Table 3. Many current methods for the detection of allelic variation are reviewed by Nollau et al, Clin. Chem. 43, 1114-1120, 1997; and in standard textbooks, for example “Laboratory Protocols for Mutation Detection”, Ed. by U. Landegren, Oxford University Press, 1996 and “PCR”, 2^{nd} Edition by Newton & Graham, BIOS Scientific Publishers Limited, 1997.

### Abbreviations:

| | |
|---|---|
| ALEX ™ | Amplification refractory mutation system linear extension |
| APEX | Arrayed primer extension |
| ARMS ™ | Amplification refractory mutation system |
| ALEX ™ | Amplification refractory mutation system linear extension |
| b-DNA | Branched DNA |
| bp | base pair |
| CMC | Chemical mismatch cleavage |
| COPS | Competitive oligonucleotide priming system |
| DGGE | Denaturing gradient gel electrophoresis |
| FRET | Fluorescence resonance energy transfer |
| HMG-CoA | 3-hydroxy-3-methylglutaryl-coenzyme A |
| LCR | Ligase chain reaction |
| MASDA | Multiple allele specific diagnostic assay |
| NASBA | Nucleic acid sequence based amplification |
| OATP | Na+-independent organic anion transporting polypeptide |
| OLA | Oligonucleotide ligation assay |
| PCR | Polymerase chain reaction |
| PTT | Protein truncation test |
| RFLP | Restriction fragment length polymorphism |
| SDA | Strand displacement amplification |
| SNP | Single nucleotide polymorphism |
| SSCP | Single-strand conformation polymorphism analysis |
| SSR | Self sustained replication |
| TGGE | Temperature gradient gel electrophoresis |

TABLE 1
Mutation Detection Techniques

| | |
|---|---|
| General: | DNA sequencing, Sequencing by hybridisation, |
| | Pyrosequencing ™ |
| Scanning: | PTT*, SSCP, DGGE, TGGE, Cleavase, Heteroduplex |
| | analysis, CMC, Enzymatic mismatch cleavage |

Hybridisation Based

| | |
|---|---|
| Solid phase | Dot blots, MASDA, Reverse dot blots, |
| hybridisation: | Oligonucleotide arrays (DNA Chips). |
| Solution phase | Taqman ™ - US-5210015 & US-5487972 |
| hybridisation: | (Hoffmann-La Roche), Molecular Beacons- Tyagi |
| | et al (1996), Nature Biotechnology, 14, 303; |
| | WO 95/13399 (Public Health Inst. New York). |
| Extension Based: | ARMS ™, ALEX ™- European Patent No. |
| | EP 332435 B1 (Zeneca Limited), COPS - Gibbs |
| | et al (1989), Nucleic Acids Research, 17, 2347. |
| Incorporation | Mini-sequencing, APEX. |
| Based: | |
| Restriction | RFLP, Restriction site generating PCR. |
| Enzyme Based: | |
| Ligation Based: | OLA. |
| Other: | Invader assay. |

*Note:
not useful for detection of promoter polymorphisms.

TABLE 2
Signal Generation or Detection Systems

| | |
|---|---|
| Fluorescence: | FRET, Fluorescence quenching, Fluorescence |
| | polarisation - United Kingdom Patent No. |
| | 2228998 (Zeneca Limited) |
| Other: | Chemiluminescence, Electrochemiluminescence, |
| | Raman, Radioactivity, Colorimetric, Hybridisation |
| | protection assay, Mass spectrometry |

TABLE 3
Further Amplification Methods

SSR, NASBA, LCR, SDA, b-DNA

Preferred mutation detection techniques include ARMS™, ALEX™, COPS, Taqman, Molecular Beacons, RFLP, and restriction site based PCR and FRET techniques.

Particularly preferred methods include ARMS™ and RFLP based methods.

Assays, for example reporter-based assays, may be devised to detect whether one or more of the above polymorphisms affect transcription levels and/or message stability.

Individuals who carry particular allelic variants of the COPD-encoding genes may therefore exhibit differences in their ability to regulate protein biosynthesis under different physiological conditions and will display altered abilities to react to COPD. In addition, differences arising as a result of allelic variation may have a direct effect on the response of an individual to drug therapy. The diagnostic methods of the invention may be useful both to predict the clinical response to such agents and to determine therapeutic dose.

Thus, in a further aspect, the methods of the invention may be used in the development of new drug therapies which selectively target one or more allelic variants of the ENaC encoding genes. Identification of a link between a particular allelic variant and predisposition to disease development or response to drug therapy may have a significant impact on the design of new drugs. Drugs may be designed to regulate the biological activity of variants implicated in the disease process whilst minimising effects on other variants.

In a further diagnostic aspect of the invention the presence or absence of variant; nucleotides is detected by reference to the loss or gain of, optionally engineered, sites recognised by restriction enzymes.

According to another aspect of the present invention there is provided an allele-specific oligonucleotide primer or an allele-specific oligonucleotide probe capable of detecting a polymorphism in a human ENaC encoding gene (or its complimentary strand), and which polymorphism preferably corresponds with one of the positions defined herein (or to a sequence complementary to such a polymorphic sequence).

Thus, the present invention provides an allele-specific oligonucleotide primer or an allele-specific oligonucleotide probe which is capable of detecting a human SCNN1B gene polymorphism at position 3870 of SEQ ID NO:1, or which is capable of detecting a SCNN1G gene polymorphism at position 10544 of SEQ ID NO:2.

According to a further feature of this aspect, the present invention provides a primer or probe which is capable of detecting a human SCNN1B gene polymorphism defined by the presence of A at position 3870 of SEQ ID NO:1. In a further embodiment, the present invention provides a primer or probe which is capable of detecting a human SCNN1B gene polymorphism defined by the presence of C at position 3870 of SEQ ID NO: 1.

According to a further feature of this aspect, the present invention provides a primer or probe which is capable of detecting a human SCNN1G gene polymorphism defined by the presence of G at position 10544 of SEQ ID NO:2. In a further embodiment, the present invention provides a primer or probe which is capable of detecting a human SCNN1G gene polymorphism which is defined by the presence of C at position 10544 of SEQ ID NO:2.

It should be noted that reference to an allele-specific oligonucleotide primer or an allele-specific oligonucleotide probe which is capable of detecting a human SCNN1B gene polymorphism at position 3870 of SEQ ED NO:1 includes an allele-specific oligonucleotide primer or an allele-specific oligonucleotide probe which is capable of detecting the compliment of a human SCNN1B gene polymorphism at position 3870 of SEQ ED NO:1; and reference to an allele-specific oligonucleotide primer or an allele-specific oligonucleotide probe which is capable of detecting a human SCNN1G gene polymorphism at position 10544 of SEQ ED NO:2 includes an allele-specific oligonucleotide primer or an allele-specific oligonucleotide probe which is capable of detecting the compliment of a human SCNN1G gene polymorphism at position 10544 of SEQ ID NO: 1.

Each primer or probe of the present invention is preferably 17-50 nucleotides in length.

The allele-specific primers of the present invention are used, generally together with a constant primer, in an amplification reaction such as a PCR reaction, which provides the discrimination between alleles through selective amplification of one allele at a particular sequence position e.g. as used for ARMS™ assays. The allele-specific primers of the present invention are preferably 17-50 nucleotides, more preferably about 17-35 nucleotides, more preferably about 17-30 nucleotides.

For example, an allele-specific primer capable of detecting a human SCNN1B gene polymorphism at position 3870 of SEQ ID NO:1 should be able to discriminate, in an amplification reaction such as a PCR reaction, between a human SCNN1B gene or a fragment thereof comprising base C at position 3870 of SEQ ID NO:1 (or a sequence complementary to such a gene or fragment), and a human SCNN1B gene or a fragment thereof comprising base A at position 3870 of SEQ JD NO:1 (or a sequence or fragment complementary to such a gene or fragment).

Furthermore, an allele-specific primer capable of detecting a human SCNN1G gene polymorphism at position 10544 of SEQ ID NO:2 should be able to discriminate, in an amplification reaction such as a PCR reaction, between a human SCNN1G gene or a fragment thereof comprising base G at position 10544 of SEQ ID NO:2 (or a sequence complementary to such a gene or fragment), and a human SCNN1G gene or a fragment thereof comprising base C at position 10544 of SEQ ID NO:2 (or a sequence complementary to such a gene or fragment).

An allele-specific primer of the present invention preferably corresponds exactly with the allele to be detected but derivatives thereof are also contemplated wherein about 6-8 of the nucleotides at the 3′ terminus correspond with the allele to be detected and wherein up to 10, such as up to 8, 6, 4, 2, or 1 of the remaining nucleotides may be varied without significantly affecting the properties of the primer.

Primers may be manufactured using any convenient method of synthesis. Examples of such methods may be found in standard textbooks, for example “Protocols for Oligonucleotides and Analogues; Synthesis and Properties,” Methods in Molecular Biology Series; Volume 20; Ed. Sudhir Agrawal, Humana ISBN: 0-89603-247-7; 1993; 1^{st} Edition. If required the primer(s) may be labelled to facilitate detection.

The allele-specific oligonucleotide probes of the present invention are preferably 17-50 nucleotides in length, more preferably about 17-35 nucleotides, more preferably about 17-30 nucleotides.

The primers and/or probes of the present invention will typically be in the form of nucleic acids (e.g. DNA or cDNA). Alternatively, the primers and/or probes may be in the form of nucleic acid analogues, for example PNA (Peptide Nucleic Acids) or LNA (Locked Nucleic Acids). The primers or probes may be nucleic acids which have been substituted in part by LNA or PNA. By employing nucleic acid analogues, specific hybridisation can be achieved with shorter oligonucleotides down to 6 bases in length.

The design of such probes will be apparent to the molecular biologist of ordinary skill. Such probes are of any convenient length such as up to 50 bases, up to 40 bases, more conveniently up to 30 bases in length, such as for example 8-25 or 8-15 bases in length. In general such probes will comprise base sequences entirely complementary to the corresponding wild type or variant locus in the gene. However, if required one or more mismatches may be introduced, provided that the discriminatory power of the oligonucleotide probe is not unduly affected. The probes of the invention may carry one or more labels to facilitate detection.

In one embodiment, an allele-specific probe capable of detecting a human SCNN1B gene polymorphism at position 3870 of SEQ ID NO:1 can discriminate, in a hybridisation is reaction, between a human SCNN1B gene or a fragment thereof comprising base C at position 3870 of SEQ ED NO:1 (or a sequence complementary to such a gene or fragment), and a human SCNN1B gene or a fragment thereof comprising base A at position 3870 of SEQ ED NO: 1 (or a sequence or fragment complementary to such a gene or fragment).

In a further embodiment, an allele-specific probe capable of detecting a human SCNN1G gene polymorphism at position 10544 of SEQ ID NO:2 can discriminate, in a hybridisation reaction, between a human SCNN1G gene or a fragment thereof comprising base G at position 10544 of SEQ ED NO:2 (or a sequence complementary to such a gene or fragment), and a human SCNN1B gene or a fragment thereof comprising base C at position 10544 of SEQ ED NO:2 (or a sequence complementary to such a gene or fragment).

According to another aspect of the present invention there is provided a diagnostic kit comprising an allele-specific oligonucleotide probe of the invention and/or an allele-specific primer of the invention.

The kit may comprise an allele-specific oligonucleotide primer capable of detecting a human SCNN1B gene polymorphism at position 3870 of SEQ ID NO:1, and an allele-specific oligonucleotide primer capable of detecting a human SCNN1G gene polymorphism at position 10544 of SEQ ID NO:2.

Alternatively, the kit may comprise an allele-specific oligonucleotide probe capable of detecting a human SCNN1B gene polymorphism at position 3870 of SEQ ID NO:1, and an allele-specific oligonucleotide probe capable of detecting a human SCNN1G gene polymorphism at position 10544 of SEQ ID NO:2.

The diagnostic kits may comprise appropriate packaging and instructions for use in the methods of the invention. Such kits may further comprise appropriate buffer(s) and polymerase(s) such as thermostable polymerases, for example taq polymerase.

According to another aspect of the present invention there is provided a method of treating a human having, or at risk of having, COPD, with a drug capable of interacting with ENaC or one of its subunits, which method comprises:
(i) detecting a polymorphism in one or more ENaC-encoding genes;
(ii) determining the status of the human by reference to the polymorphism(s); and
(iii) administering an effective amount of the drug.

According to another aspect of the present invention there is provided a method of treating a human having, or at risk of having, COPD, with a drug capable of treating COPD, which method comprises:
(i) detecting a polymorphism in one or more ENaC-encoding genes;
(ii) determining the status of the human by reference to the polymorphism(s); and
(iii) administering an effective amount of the drug.

Preferably, in each of these aspects, the polymorphism is a single nucleotide polymorphism, and preferably the one or more genes are human SCNN1B and/or human SCNN1G. The polymorphism is preferably at one or more positions defined herein. The methods can also involve detecting two or more of the polymorphisms defined herein. When the methods comprise detecting a polymorphism in SCNN1B, they preferably comprise determining the nucleotide sequence of the individual at position 3870 of SEQ ID NO: 1; and preferably still, detecting the presence of base A at position 3870 of SEQ ID NO:1. When the methods comprise detecting a polymorphism in SCNN1G, they preferably comprise determining the nucleotide sequence of the individual at position 10544 of SEQ ED NO:2; and preferably still, comprise detecting the presence of base G at position 10544of SEQ ID NO:2.

Examples of drugs which can be used for the treatment of COPD include beta-agonists, and in particular beta-2-agonists (such as formoterol and salmeterol), anticholinergics (such as tiotropium), theophylline, N-acetylcysteine, a combination of a long-acting beta-agonist and an inhaled corticosteroid (such as the combination of formoterol and budesonide, or the combination of fluticasone and salmeterol), and a combination of an anticholinergic and albuterol (such as the combination of albuterol and ipratropium). It should be noted that reference to the above compounds includes pharmaceutically acceptable salts or solvates thereof. For example, the term “formoterol” encompasses the free base as well as e.g. formoterol fumarate dehydrate.

According to another aspect of the invention there is provided the use of a drug capable of interacting with ENaC or one of its subunits in the preparation of a medicament for treating an individual for COPD, wherein the individual has been identified as having a polymorphism which is associated with COPD in one or more ENaC encoding genes.

The present invention also provides the use of a drug or drug combination selected from the group consisting of use of a drug or drug combination selected from the group consisting of a beta-agonist, an anticholinergic, theophylline, N-acetylcysteine a combination of a long-acting beta-agonist and an inhaled corticosteroid, and a combination of an anticholinergic and albuterol, in the preparation of a medicament for treating COPD in a human determined as having a polymorphism in one or more ENaC-encoding genes. Preferably, the polymorphism is a single nucleotide polymorphism, and preferably the one or more genes are human SCNN1B and/or human SCNN1G. The polymorphism(s) are preferably at one or more positions defined herein.

Further examples of suitable drugs which can be employed in the present invention are antisense molecules (which can be targeted against the mRNA of ENaC-encoding genes) or an antibody or antibody derivative directed against ENaC or one of its subunits or a homologue thereof. The preparation of antibodies and antisense molecules are well know in the art.

As used herein the term antibody is to be understood to mean a whole antibody or a fragment thereof, for example a F(ab)2, Fab, FV, VH or VK fragment, a single chain antibody, a multimeric monospecific antibody or fragment thereof, or a bi- or multi-specific antibody or fragment thereof. Each of these types of antibody derivative and their acronyms are well known to the person skilled in the art. Methods of making and detecting labelled antibodies are well known (Campbell; Monoclonal Antibody Technology, in: Laboratory Techniques in Biochemistry and Molecular Biology, Volume 13. Eds: Burdon R et al. Elsevier, Amsterdam (1984)). The term antibody includes both monoclonal antibodies, which are a substantially homogeneous population, and polyclonal antibodies which are heterogeneous populations. The term also includes inter alia, humanised and chimeric antibodies. Monoclonal antibodies to specific antigens may be obtained by methods known to those skilled in the art, such as from hybridoma cells, phage display libraries or other methods. Monoclonal antibodies may be inter alia, human, rat or mouse derived. For the production of human monoclonal antibodies, hybridoma cells may be prepared by fusing spleen cells from an immunised animal, e.g. a mouse, with a tumour cell. Appropriately secreting hybridoma cells may thereafter be selected (Koehler & Milstein, Nature 256:495-497 (1975); Cole et al., “Monoclonal antibodies and Cancer Therapy”, Alan R Liss Inc, New York N.Y. pp 77-96 (1985)). Such antibodies may be of any immunoglobulin class including IgG, IgM, IgE, IgA, IgD and any subclass thereof. Polyclonal antibodies can be generated by immunisation of an animal (such as a mouse, rat, goat, horse, sheep etc) with an antigen, such as one of the FGF-BP1 proteins used in this invention.

By the term drug interacting with ENaC or one of its subunits is meant a drug which affects the functional activity of ENaC or one of its subunits, or the expression thereof. The term interacting in the context of the present invention is synonymous with modulating, and may include any one or more of the following: conformational change, covalent modification, or inhibition. Modulators include inhibitors (such as antagonists). Modulation of ENaC or one of its subunits by a compound may be brought about, for example, through compound binding to ENaC or one of its subunits. The term modulates and modulating should be construed accordingly.

As mentioned above, the present invention identifies an association of ENaC-encoding genes with the respiratory disease COPD. The present invention therefore identifies a is functional role for ENaC, and in particular, the subunits encoded by SCNN1B and SCNN1G.

Accordingly, in another aspect of the invention there is provided a method for treating COPD in an individual, which method comprises modulating the expression of ENaC or one or more of its subunits (e.g. SCNN1B and/or SCNN1G) or modulating functional activity of ENaC or one or more of its subunits (e.g. the protein encoded by SCNN1B and/or SCNN1G).

In another aspect of the invention there is provided an assay for screening for and identifying a compound as a potential compound that modulates the function of ENaC or one of its subunits (e.g. the protein encoded by SCNN1B and/or SCNN1G), and which can be used for the treatment of COPD.

Whether a given drug/compound/agent interacts with, or modulates ENaC or one of its subunits can be determined, for example, by the following methods:
by functional assays of ENaC or one or more of its subunits, to determine whether its activity is modulated;
by direct measurement of the binding or interaction of the compound with ENaC or one or more of its subunits (including competitive binding assays);
by immunological assays (for example, using an antibody specific for ENaC or one or more of its subunits to determine whether protein levels of ENaC or one of its subunits are affected);
by assays to determine whether gene expression of one or more of the ENaC-encoding genes is affected.

The invention will now be illustrated but not limited by reference to the following Examples.

### EXAMPLE 1

### Identification and Analysis of SNPs

A map of SNPs was constructed for human SCNN1A, human SCNN1B and human SCNN1G.

Each map comprised the nucleic acid sequence of each gene plus annotations which indicated the presence of exons, introns, and known or predicted polymorphisms. The maps were made using an electronic laboratory which gathers nucleic acid sequences from the EnsEMBL Human Genome Server and assigns exons and polymorphisms to the sequences. The polymorphisms assigned are those which are accessible in the public SNP databases and includes the TSC (the SNP consortium), the NCBI (the National Center of Biotechnology Information), and the EBI (European Bioinformatics Institute); polymorphisms present in either of ABI's (Applied biosystems) databases including Assays on Demand and its Virtual SNP database; as well as polymorphisms present in the published literature or otherwise known.

One of the SNPs identified was present in the NCBI database (found at http://www.ncbi.nlm.nih.gov/SNP/) under ID number rs63982. This SNP corresponds to that identified at position 3870 of SEQ ED NO:1 of the present application (with nucleotide base A or variant nucleotide base C at this position).

Another of the SNPs identified was present in the NCBI database (found at http://www.ncbi.nlm.nih.gov/SNP/) under ED number rs11643777. This same SNP was also present in ABI's (Applied biosystems) Assays on Demand web site under name C_{—}191762_{—}10. This SNP corresponds to that identified at position 10544 of SEQ ID NO:2 of the present application (with nucleotide base C or variant nucleotide base G at this position).

Many of the polymorphisms assigned to the gene sequence maps were confirmed or validated as true polymorphic sites by sequencing nucleic acid extracted from a panel of lymphopblastoid cell lines. This validation procedure was performed by designing PCR primers spanning the positions of database SNPs, and then using these primers to amplify segments of DNA from 15 distinct human cell lines. The PCR products generated were then subjected to DNA sequencing and the resulting sequence traces examined for sequence variation along their entire lengths. This process was used to validate the presence of polymorphisms predicted from the SNP databases and also identify novel sites of polymorphic variation.

For the disease-associated SNP in SCNN1B that corresponds to position 3870 of SEQ ID NO:1, primers were designed that spanned exon 3, capturing a small amount of intronic sequence on either side. The nucleic acid sequence captured by these primers also included the position of the database SNP rs63982 (identified by position 3870 in SEQ ED NO:1). The sequences of these primers were: 5′-ACCCAGTCTCAGGTAGTATC-3′ (SEQ ID NO:3) and 5′-CCAGCGAGACTCAAATTAC-3′ (SEQ ID NO:4). In an appropriate reaction containing Taq DNA polymerase, buffers, magnesium chloride, and human genomic DNA, the primers (noted above) generated a product of 492 bp. DNA sequencing of this product, generated from 15 different cell lines, confirmed the presence of SNP rs63982.

For the disease associated SNP in SCNN1G, that corresponds to position 10544 of SEQ ID NO:2, a working genotyping assay for this SNP was available in ABI's Assays on Demand database.

### Genotyping SNPs in COPD Patients

In total, 44 SNPs in 3 ENaC genes were chosen for genotyping in a sample of approximately 700 patients with COPD and 450 smoking-matched controls. Sixteen of the 44 SNPs were chosen from ABI's Assays on Demand SNP database.

For the 16 “Assays on Demand” SNPs, oligonucleotide reagents specific for each SNP, together with a common reaction master mix were purchased from ABI. The purchased Assays on Demand reagents comprise optimised oligonucleotides that specifically detect a single named SNP. For the remaining 28 SNPs, custom primers were prepared to detect these SNPs. The genotyping methodology employed was the Taqman allelic discrimination assay. PCR primers were chosen to amplify a small segment of nucleic acid containing the SNP of interest. Included in the amplification reactions were two oligonucleotides probes, each one specific to one allele of the SNP. During the course of amplification, each probe hybridised to its target allele, generating fluorescence that was quantitated by a sensitive detector. Since each of the two probes was labelled with a different fluorochrome, usually FAM and VIC, the presence of one or both alleles in patient or control DNA could be determined, and captured electronically. Since each SNP is biallelic, 3 different genotypes are possible, so for a SNP with alleles C and G, the 3 different genotypes are CC, CG and GG. In the Taqman allelic discrimination assay, such a SNP could be genotyped if the C allele were hybridised by a probe labelled with the fluorochrome FAM and the G allele hybridised with a probe labelled with the fluorochrome VIC. In this example, the CC genotype would be characterised by FAM fluorescence only; the CG genotype by FAM and VIC fluorescence; and the GG genotype by VIC fluorescence only. In this way, each of the 44 ENaC SNPs were genotyped.

Details of the SNP rs63982, corresponding to that identified at position 3870 of SEQ ID NO:1 are as follows:

| | |
|---|---|
| Forward PCR primer | GAGTGGGTCCCAGATTTCATT |
| | (SEQ ID NO:5) |
| Reverse PCR primer | GGTCCTTCAGCAAATGCTTGATT |
| | (SEQ ID NO:6) |
| VIC probe | TGCTTTACAGCTATGCAT |
| (detects allele C) | (SEQ ID NO:7) |
| FAM probe | CTGCTTTACAGATATGCAT |
| (detects allele A) | (SEQ ID NO:8) |

The COPD patients and controls were from throughout Europe. A diagnosis of COPD was confirmed by respiratory physicians and all patients (cases) had mild disease. The matching of cases and controls for geographical location as well as smoking increases the utility of this DNA collection for genetic studies by reducing the chance of a false positive result related to population substructure, and controlling for known environmental risk factors for COPD. The DNA collection is also appropriately sized to detect a genetic effect due to a susceptibility allele.

The genotyping data for each of the SNPs was analysed for genetic association to COPD in two ways. In the first, the number of individuals with each of the three possible genotypes for each SNP was compared between cases and controls. Chi-squared tests were performed which compared the observed distributions with those expected if there was no association. For each SNP, a p value was generated which is the probability of the observed result due to chance. A p value equal to, or less than, 0.05 was taken as evidence of genetic association between a single SNP and COPD.

The second analysis method compared the allele frequencies for each SNP between cases and controls. This allele-wise method is not sensitive to deviations in Hardy-Weinberg equilibrium like that occasionally seen with the genotype-based method described above. In the allele-wise method, odds ratios were calculated for each SNP, which are a measure of the odds of disease in the presence of one allele over the odds of disease in the absence of that allele. For each SNP a p value was calculated which is a measure of the confidence in the odds ratio.

Evidence for genetic association to COPD was found for two of the genes examined. In SCNN1B, one SNP in particular was strongly associated with COPD, as evidenced by genotype and allele-wise p values of less than 0.001. This SNP was identified by the NCBI identifier rs63982 and corresponds to position 3870 of SEQ ID NO:1. The evidence for genetic association of this SNP to COPD is shown in Table 1 below. The data indicates that the “A” allele of SNP rs63982 is increased in COPD cases compared with controls.

TABLE 1
Genotype-wise test for association

| | Genotype | | | |
|---|---|---|---|---|
| | CC | CA | AA | |
| Controls | 29 | 171 | 247 | X² = 16.88 |
| Cases | 17 | 223 | 441 | p = 0.0002 |

Allele-wise test for association

| | Freq. allele C | Freq. allele A | |
|---|---|---|---|
| Controls | 25.6% | 74.4% | ODDS = 1.48 |
| Cases | 18.9% | 81.1% | p = 0.0001 |

Support for the genetic association of the SCNN1B SNP at position 3870 of SEQ ID NO:1 comes from an analysis of the position of the SNP in the gene sequence. The SNP occurs in close proximity to exon 3 and may impact gene function by interfering with mRNA expression or splicing. The possible functional consequence of the associated SNP, in altering SCNN1B gene expression either quantitatively or qualitatively, adds credence to the observed disease association.

Evidence for genetic association to COPD was also found for SCNN1G, in which a single SNP demonstrated a positive correlation with COPD. This positive SNP was identified by the NCBI identifier rs11643777 and corresponds to position 10544 of SEQ ID NO:2. The evidence for genetic association of this SNP to COPD is shown in Table 2 below. The data indicates that the “G” allele of SNP rs11643777 is increased in COPD cases compared with controls.

TABLE 2
Genotype-wise test for association

| | Genotype | | | |
|---|---|---|---|---|
| | CC | CG | GG | |
| Controls | 347 | 95 | 7 | X² = 5.44 |
| Cases | 487 | 178 | 18 | p = 0.0660 |

Allele-wise test for association

| | Freq. allele C | Freq allele G | |
|---|---|---|---|
| Controls | 87.9% | 12.1% | ODDS = 1.34 |
| Cases | 84.3% | 15.7% | p = 0.0192 |

1. A method for assessing the predisposition and/or susceptibility of an individual to COPD, which method comprises detecting the presence of a polymorphism in one or more human ENaC-encoding genes.

2. A method according to claim. 1 wherein the polymorphism is a single nucleotide polymorphism.

3. A method according to claim 1, wherein the one or more genes are human SCNN1B and/or SCNN1G.

4. A method according to claim 3, which comprises detecting the presence of a polymorphism in human SCNN1B.

5. A method according to claim 4, wherein the method comprises determining the nucleotide of the individual at position 3870 of SEQ ID NO: 1.

6. A method according to claim 5, wherein the method comprises detecting the presence of A and/or C at position 3870 of SEQ ID NO:1.

7. A method according to claim 3, which comprises detecting the presence of a polymorphism in SCNN1G.

8. A method according to claim 7, wherein the method comprises determining the nucleotide of the individual at position 10544 of SEQ ID NO:2.

9. A method according to claim 8, wherein the method comprises detecting the presence of C and/or G at position 10544 of SEQ ID NO:2.

10. An allele-specific oligonucleotide primer capable of detecting an SCNN1B gene polymorphism at position 3870 of SEQ ID NO: 1.

11. An allele-specific oligonucleotide probe capable of detecting an SCNNIB gene polymorphism, at position 3870 of SEQ ID NO:1.

12. A primer as defined in claim 10, which is capable of detecting either (i) the presence of A at position 3870 of SEQ ID NO:1, or (ii) the presence of C at position 3870 of SEQ ID NO:1.

13. A primer as defined in claim 10 which is in the range of 17-50 nucleotides in length.

14. A diagnostic kit comprising an allele-specific oligonucleotide probe as defined in claim 11.

15. A diagnostic kit according to claim 14, and further comprising an allele-specific primer capable of detecting an SCNN1G gene polymorphism at position 10544 of SEQ ID NO:2 and/or an allele specific oligonucleotide probe capable of defecting an SCNN1G gene polymorphism at position 10544 of SEQ ID NO:2.

16. A diagnostic kit according to claim 15, wherein the further allele-specific primer or probe is either (i) capable of detecting the presence of G at position 10544 of SEQ ID NO:2, or (ii) is capable of detecting the presence of C at position 10544 of SEQ ID NO:2.

17. A diagnostic kit according to claim 15 wherein the allele-specific primer or probe capable of detecting an SCNN1G gene polymorphism at position 10544of SEQ ID NO:2 is in the range of 17-50 nucleotides in length.

18. A method of treating a human having, or at risk of having, COPD, with a drug capable of interacting with human ENaC or one of its subunits, which method comprises:
(i) detecting a polymorphism in one or more human ENaC-encoding genes;
(ii) determining the status of the human by reference to the polymorphism(s); and
(iii) administering an effective amount of the drug.

19. A method of treating a human having, or at risk, of having, COPD, with a drag capable of treating COPD or one of its subunits, which method comprises:
(i) detecting a polymorphism in one or more human ENaC-encoding genes;
(ii) determining the status of the human by reference to the polymorphism(s); and
(iii) administering an effective amount of the drug.

20. A method according to claim 19, wherein the drug is selected from the group consisting of a beta-agonist, an anticholinergic, theophylline, N-acetylcysteine, a combination of a long-acting beta-agonist and an inhaled corticosteroid, or a combination of an anticholinergic and albuterol.

21. A method according to claim 18, wherein the polymorphism is a single nucleotide polymorphism.

22. A method according to claim 18, wherein the one or more genes are human SCNN1B and/or human SCNN1G.

23. A method according to claim 22, wherein the gene is human SCNN1B.

24. A method according to claim 23, wherein the method comprises determining the nucleotide of the individual at the position corresponding to position 3870 of SEQ ID NO; 1.

25. A method according to claim 24, wherein the method comprises detecting the presence of base A at the position corresponding to position 3870 of SEQ ID NO:1.

26. A method according to claim 22, wherein the gene is human SCNN1G.

27. A method according to claim 26, wherein the method comprises determining the nucleotide of the individual at position 10544 of SEQ ID NO:2.

28. A method according to claim 27, wherein the method comprises detecting the presence of base G at position 10544 of SEQ ID NO:2.

29. Use of a drag capable of interacting with human ENaC or one of its subunits in the preparation of a medicament for treating COPD in a human determined as having a polymorphism in one or more ENaC-encoding genes.

30. Use of a drug selected from the group consisting of a beta-agonist, an anticholinergic, theophylline, N-acetylcysteine, a combination of a long-acting beta-agonist and an inhaled corticosteroid, or a combination of an anticholinergic and albuterol, in the preparation of a medicament for treating COPD in a human determined as having a polymorphism in one or more ENaC-encoding genes.

31. Use according to claim 29, wherein the polymorphism is at a position corresponding to any one of the following; position 3870 of SEQ ID NO:1 and position 10544 of SEQ ID NO:2.

32. Use according to claim 31 wherein the Iranian is determined as having a base A at position 3870 of SEQ ID NO: 1 and/or base G at position 10544 of SEQ ID NO:2.
